(19) United States
(12) Patent Application Publication
(10) Pub. No.: US 2022/0053848 A1
(43) Pub. Date: Feb. 24, 2022

(54) DECOMPRESSION FLOATING PROTECTIVE PATELLAR KNEE JOINT PAD

(71) Applicants: Jeffrey Matthews, Half Moon Bay, CA (US); Lori Matthews, Half Moon Bay, CA (US); Daniel K. Chisholm, Emerald Hills, CA (US)

(72) Inventors: Jeffrey Matthews, Half Moon Bay, CA (US); Lori Matthews, Half Moon Bay, CA (US); Daniel K. Chisholm, Emerald Hills, CA (US)

(21) Appl. No.: 17/396,480

(22) Filed: Aug. 6, 2021

Related U.S. Application Data
(60) Provisional application No. 63/069,566, filed on Aug. 24, 2020.

(51) Int. Cl. A41D 13/06; A41D 31/14; A41D 31/18; A63B 71/12

(52) U.S. Cl. CPC A41D 13/065; A41D 31/145; A41D 2300/32; A63B 71/1225; A63B 2071/125; A41D 31/185

(57) ABSTRACT

A decompression floating protective patellar knee joint pad for preventing contact between the patella and an external surface may include a fabric sleeve sized to surround a user's knee region, extending from the user's lower leg to the user's thigh; a stabilizing platform with a central opening mimicking a shape of the patellar region of the user attached to the fabric sleeve; a cushioning material attached to a posterior surface of the stabilizing; and a patellar cap attached to the stabilizing platform and covering the central opening, wherein the patellar cap is protracted away from the user, creating a void configured to prevent the patella from having contact with the external surface.

### RELATED APPLICATION

This application claims priority to provisional patent application U.S. Ser. No. 63/069,566 filed on Aug. 24, 2020, the entire contents of which is herein incorporated by reference.

### BACKGROUND

The embodiments described herein relate generally to medical devices and, more particularly, to a floating decompression patellar knee pad.

Existing knee pads have constant contact with the patella, causing compression and, thus, pressure on the patellar region and associated ligaments and cartilages, with weight bearing on the knee joint. This can cause pain and discomfort during use, particularly for individuals with existing or prior knee injuries.

Therefore, what is needed is a knee pad that does not contact the patella, thus minimizing compression on the patella and knee joint and transferring force and weight from kneeling to the distal aspect of the femoral region and the proximal aspect of the tibia, fibular, and tibial regions.

### SUMMARY

Some embodiments of the present disclosure include a decompression floating protective patellar knee joint pad for preventing contact between the patella and an external surface may include a fabric sleeve sized to surround a user's knee region, extending from the user's lower leg to the user's thigh; a stabilizing platform with a central opening mimicking a shape of the patellar region of the user attached to the fabric sleeve; a cushioning material attached to a posterior surface of the stabilizing platform along edges of the central opening, the cushioning material including an opening concentrically aligned with the central opening; and a patellar cap attached to the stabilizing platform and covering the central opening, wherein the patellar cap is protracted away from the user, creating a void configured to prevent the patella from having contact with the external surface. The structure of the knee pad may provide for protection of the patella, patellar/femoral tendons, and the knee joint from blunt forces in sports, kneeling, and falls because the patella is prevented from contacting an external surface. Moreover, the energy or force to the knee joint and patella may be transferred to the distal part of the femur and proximal part of the lower leg, hence leaving the patella untouched.

### BRIEF DESCRIPTION OF THE FIGURES

The detailed description of some embodiments of the invention is made below with reference to the accompanying figures, wherein like numerals represent corresponding parts of the figures.

FIG. 1 is a front elevation view of one embodiment of the present disclosure, shown in use.

FIG. 2 is a side elevation view of one embodiment of the present disclosure, shown in use.

FIG. 3 is a cross-sectional view of one embodiment of the present disclosure, taken along line 3-3 in FIG. 1.

FIG. 4 is a rear elevation view of one embodiment of the present disclosure.

FIG. 5 is a rear elevation view of one embodiment of the present disclosure.

FIG. 6 is a front elevation view of the anatomy of a human knee.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used as a decompression knee pad and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

The various elements of the present disclosure may be related in the following exemplary fashion. It is not intended to limit the scope or nature of the relationships between the various elements and the following examples are presented as illustrative examples only.

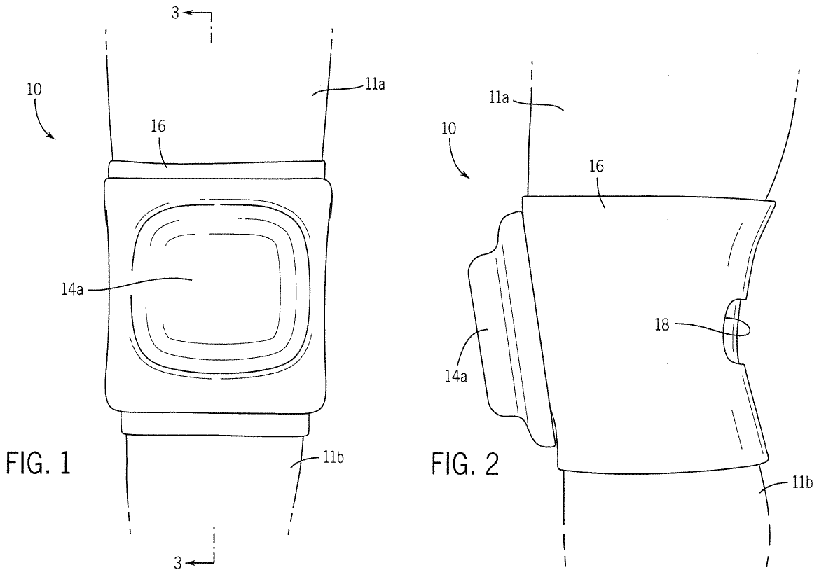

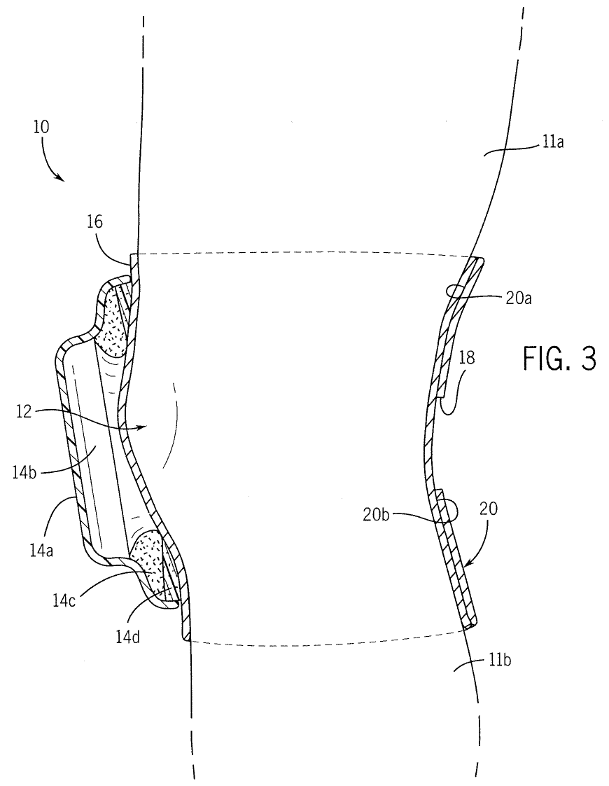

By way of example, and referring to FIGS. 1-5, some embodiments of the invention include a floating decompression patellar knee pad for preventing contact by the patella to any surface and transferring compression from a concave patellar cap to the medial/lateral/anterior regions of the distal femoral and proximal regions of the tibia tuberosity, medial condyle of the tibia, Gerdy's tubercle, and the head of the fibula and proximal regions of the tibia bone without causing compression or any type of physical force on the superior/inferior, lateral/medial, and anterior/posterior aspect of the patella, including the patellar/femoral tendon. At a top level, embodiments of the knee pad of the present disclosure may comprise a platform with an opening covered by a concave cap that surrounds the patella and the anterior knee joint region to prevent any physical contact with the patella and the anterior knee joint.

More specifically, as shown in FIGS. 1-5, a first embodiment of the knee pad 10 may comprise a fabric sleeve 16 sized to surround the knee region of a user extending from the thigh 11a to the lower leg 11b, wherein a rear portion of the sleeve 16 (i.e. a portion of the sleeve 16 designed to be positioned against the back of a user's knee) may include an opening extending there through; a stabilizing platform attached to a front portion of the sleeve 16 (i.e., a portion of the sleeve 16 designed to be positioned proximate to the user's patellar region 12), wherein the stabilizing platform comprises a layered construction comprising an inner memory foam layer 14d and an outer cushioning gel layer 14c, the inner memory foam layer 14d positioned against the fabric sleeve 16, and wherein each of the inner memory foam layer 14d and outer cushioning gel layer 14c have a central opening sized to accommodate extension of the user's knee cap therein; and a patellar cap 14a attached to and extending outward from the outer surface of the stabilizing platform, creating a void 14b in fluid communication with the central opening to prohibit the patella from any contact with an external surface. In some embodiments, the patellar cap 14a is concave and protracted away from the user to create such void. Moreover, the sleeve 16 may comprise the rear opening 18 to prevent the wrap from putting pressure on the user's popliteal fossa.

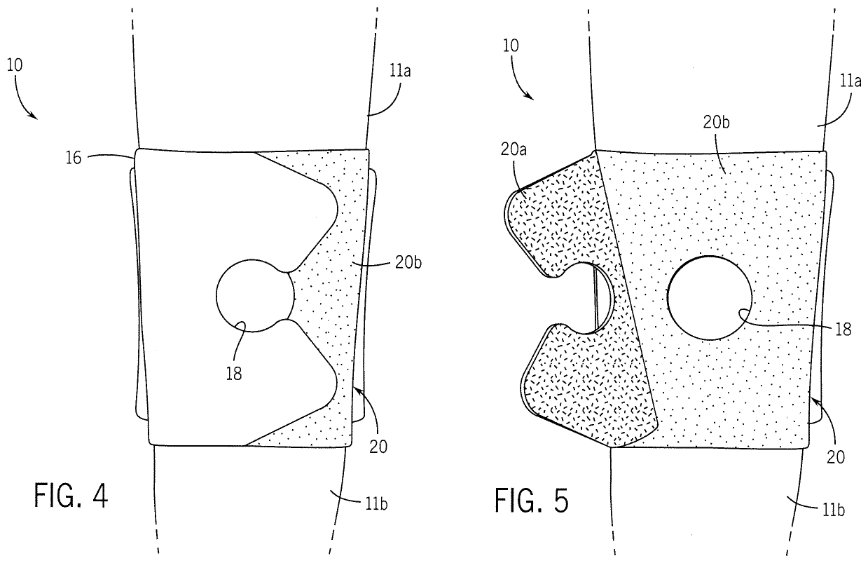

As shown in the Figures, the fabric sleeve 16 may comprise a hook and loop fastener 20 for adjustability in the size thereof. For example, an outer surface of the posterior portion of the sleeve 16 may comprise a loop surface 20b, while an adjustment wrap that extends outward from the sleeve 16 may comprise an inner hook surface 20a. The hook surface 20a may be designed to removably engage with the loop surface 20b, as shown in FIG. 4, to secure the knee pad around a user's leg. Thus, in embodiments, the adjustable fabric sleeve 16 may comprise a wrap-like structure.

The device of the present disclosure may be made of any suitable materials and may have any desired or necessary dimensions. It is envisioned that the knee pad may be produced in various sizes, such as small, medium, large, and extra-large to accommodate various sized users. In a particular embodiment, the stabilizing platform may comprise a semi-rigid material, such as soft plastic, while the patellar cap may comprise a thin layer of a more rigid plastic to prevent injury to the patellar region 12 by uneven surfaces. The opening in the stabilizing platform may be substantially rectangular to accept the prominences of the four corners of the knee complex. Moreover, the cushioning gel may comprise a material that provides for comfort of the supporting structures of the distal thigh and proximal lower leg. The fabric sleeve may comprise a breathable, yet strong and flexible material, such as an elasticized material. The memory foam may be designed and positioned to conform to the distal femoral and proximal regions of the tibia, fibula, and tibial regions and help prevent the knee pad from slipping down the lower leg.

In some embodiments, the cushioning gel may be adhered to a posterior surface of the stabilizing platform and a layer of memory foam may be adhered to a posterior surface of the cushioning gel, wherein the posterior surface is a surface of the stabilizing platform/cushioning gel that is designed to face or be oriented toward the user when the knee pad is being worn. Together, these materials may provide a structure that provides comfort and that allows the distal portion of the femur and the proximal part of the tibiofibular regions to conform to the distal part of the thigh and the proximal part of the lower leg.

Due to its structure, the patella and protective knee pad of the present disclosure may protect the following structures in the human body: patellar ligament, tibial collateral ligament, quadriceps femoris tendon, infrapatellar fat pad, subcutaneous prepatellar bursa, suprapatellar (synovial) bursa, subcutaneous infrapatellar bursa, anterior aspect of the medial/lateral meniscus, anterior aspect of the medial/lateral condyle of the femur, tibial collateral ligament, lateral patellar retinaculum, medial patellar retinaculum, bursa deep to iliotibial tract, insertion of iliotibial tract to Gerdy's tubercle and oblique line of tibia, Vastus medialis/Vastus lateralis muscles, joint capsule, synovial membrane, anterior cruciate ligament, medial condyle of tibia, head of fibula, and infrapatellar synovial fold.

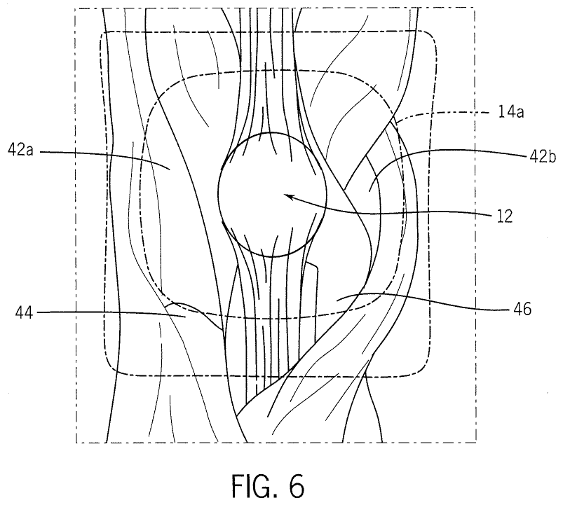

To use the device of the present disclosure, a user may put the decompression patellar knee joint pad on his or her knee and secure. Correct placement of the patellar cap and stabilizing platform with relation to the patellar region 12, lateral epicondyle of the femur 42a, medial epicondyle of the femur 42b, head of the tibia 44, and medial condyle of the tibia 46 is shown in FIG. 6. The knee pad may inhibit contact of the patella and the anterior knee joint and any physical surface that the pad comes into contact with. Thus, the decompression knee pad may eliminate any physical contact with the patella and knee joint. As such, the decompression knee pad may prevent general knee pain from kneeling, chondromalacia, pre-bursitis, bursitis, arthritis of the knee joint and patella, patellar femoral tendonitis, patellar dislocations, and low and high impact injuries, such as fractures of the patella and knee joint. Due to the structure of the device, the user's weight when kneeling may be transferred to the static regions of the distal femur and proximal regions of the tibia/fibular and tibia bone. In effect, the patellar/femoral tendon, patella, and knee joint may have no contact with the patellar cap or the stabilizing platform. Specifically, the gel, memory foam, stabilizing platform, and patellar cap together may prohibit the patella and knee joint from any harm from external forces.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

What is claimed is:

1. A decompression floating protective patellar knee joint pad for preventing contact between the patella and an external surface, the decompression floating protective patellar knee joint pad comprising:
a fabric sleeve sized to surround a user's knee region, extending from the user's lower leg to the user's thigh;
a stabilizing platform with a central opening mimicking a shape of the patellar region of the user attached to the fabric sleeve;
a cushioning material attached to a posterior surface of the stabilizing platform; and
a patellar cap attached to the stabilizing platform and covering the central opening, wherein the patellar cap is protracted away from the user, creating a void configured to prevent the patella from having contact with the external surface.

2. The decompression floating protective patellar knee joint pad of claim 1, wherein the cushioning material comprises:
a layer of cushioning gel attached to a posterior surface of the stabilizing platform; and
a layer of foam attached to a posterior surface of the cushioning gel.

3. The decompression floating protective patellar knee joint pad of claim 2, wherein the layer of foam comprises memory foam.

4. The decompression floating protective patellar knee joint pad of claim 1, wherein the fabric sleeve comprises a rear opening extending configured to prevent pressure being out on the user's popliteal fossa by the fabric sleeve.

5. The decompression floating protective patellar knee joint pad wherein the patellar cap is concave.

6. The decompression floating protective patellar knee joint pad of claim 1, wherein the fabric sleeve comprises a breathable and stretchable material.

7. The decompression floating protective patellar knee joint pad of claim 1, wherein the fabric sleeve comprises a wrap comprising a hook and loop fastener.
